(19) United States
(12) Patent Application Publication
(10) Pub. No.: US 2007/0010726 A1
(43) Pub. Date: Jan. 11, 2007

(54) Internal biochemical sensing device

(75) Inventors: Gerald E. Loeb, South Pasadena, CA (US); Thomas George, La Canada, CA (US); Cesar Blanco, Los Angeles, CA (US); Laura Marcu, Sierra Madre, CA (US)

Correspondence Address: McDERMOTT WILL & EMERY, LLP;34th Floor, 2049 Century Park East, Los Angeles, CA, 90067, US

(73) Assignee: Alfred E. Mann Inst. for Biomedical Engineering at the University of Southern California

(21) Appl. No.: 11/351,158

(22) Filed: Feb. 8, 2006

Related U.S. Application Data
(63) Continuation-in-part of application No. 10/263,272, filed on Oct. 2, 2002, now Pat. No. 7,096,053.
(60) Provisional application No. 60/651,318, filed on Feb. 9, 2005.

(51) Int. Cl. A61B 5/00

(52) U.S. Cl. 600/317; 600/342

(57) ABSTRACT

A biosensing device for detecting biological analytes, and methods of use and manufacture, are disclosed. The device includes a biosensing element that can remain implanted for extended periods of time. The biosensing element is connected to an optical fiber terminating outside of the body. The optical fiber is also connected to an information analyzer. The information analyzer receives light from the reaction of fluorescent molecules in the biosensing element. The biosensing device can be used to detect and analyze the effectiveness of chemotherapy agents and molecules associated with various diseases.

### CROSS REFERENCE TO RELATED APPLICATIONS

This United States Patent Application is a continuation-in-part of U.S. patent application Ser. No. 10/263,272, filed Oct. 2, 2002, entitled “Internal Biochemical Sensing Device;” and claims the benefit of the filing date of U.S. provisional patent application Ser. No. 60/651,318, filed Feb. 9, 2005, entitled “Internal Biochemical Sensing Device;” the contents of each of which are incorporated herein by reference.

### BACKGROUND OF THE INVENTION

1. Field of the Invention

The present invention relates generally to implanted devices and methods for detection of biochemical analytes.

2. General Background and State of the Art

In order to detect or manage certain diseases or conditions, it is useful to make frequent measurements of specific biochemical analytes, hereinafter referred to as “analytes,” within a patient's body over an extended period of time. For example, glucose levels in a patient's body can be monitored to guide the dosage of insulin required to treat diabetes mellitus. Another example would be monitoring the tissue concentration of therapeutic drugs such as anticoagulants, immunosuppressive agents and anticancer drugs, all of which can lead to serious complications if the tissue levels are too high or too low. A further example includes monitoring the effectiveness of therapeutic agents, such as chemotherapy agents, on targeted cells.

Several chemotherapeutic agents cause cancer cells to undergo programmed cell death, also known as apoptosis. Once initiated, apoptosis leads to a cascade of biochemical and morphological events that cause irreversible degradation of genomic DNA and fragmentation of the cell. Apoptosis causes the expression of specific genes that encode for proteins involved in the cascade of various biochemical and morphological events.

Often chemotherapeutic agents destroy normal cells in addition to cancer cells. In many current chemotherapy regimens, the goal is to kill as many cancer cells as possible while minimizing collateral damage to healthy cells. As cancer appears in many different forms, chemotherapies destroy cancer cells with varying levels of success among individual patients. To determine whether particular chemotherapy agents and regimens are effectively killing cancer cells, physicians typically administer the chemotherapy agents for enough time to kill enough cancer cells to allow imaging devices to show reductions in tumor size. In addition, in vitro testing of blood or tissue samples can be used to determine whether chemotherapies are destroying cancer cells.

However, current methods for detecting the effects of chemotherapies on cancer cells can include prolonged waiting times in order to allow the chemotherapies to have enough time to allow adequate detection of cancer cell death by common imaging or in vitro diagnostics of blood samples. During this waiting period, valuable treatment time may be lost and unnecessary damage to healthy patient cells may ensue. Moreover, when several therapeutic agents are administered simultaneously or in rapid succession, it may be impossible or impractical to use current diagnostic devices and methods to determine in a relatively rapid manner the respective degrees of effectiveness of the various therapeutic agents.

### SUMMARY

One aspect of the biochemical sensing devices comprises an optical fiber having a first end, a second end configured to connect to an analyzer, and a biosensing material attached to the first end comprising a polymer matrix containing tubulin covalently bound to the polymer matrix, and capable of interacting with a chemotherapeutic agent.

Another aspect of the biochemical sensing devices comprises an optical fiber having a first end, a second end configured to connect to an analyzer, and a biosensing material attached to the first end comprising a polymer matrix and at least at least one fluorescent molecule covalently bound to the polymer matrix that can fluoresce upon interaction with at least one molecule indicative of apoptosis.

It is understood that other embodiments of the present biochemical sensing devices and methods will become readily apparent to those skilled in the art from the following detailed description, wherein it is shown and described only exemplary embodiments by way of illustration. As will be realized, the biochemical sensing devices and methods are capable of other and different embodiments and its several details are capable of modification in various other respects. Accordingly, the drawings and detailed description are to be regarded as illustrative in nature and not as restrictive.

### BRIEF DESCRIPTION OF THE DRAWINGS

Aspects of the present invention are illustrated by way of example, and not by way of limitation, in the accompanying drawings, wherein:

FIG. 1 illustrates a compact and portable biosensing device and an exemplary mode of positioning relative to a patient's body;

FIG. 2 illustrates an exemplary biosensing element configuration;

FIG. 3 is a functional block diagram of an exemplary information analyzer;

FIG. 4 illustrates an exemplary information analyzer configuration;

FIG. 5 illustrates an exemplary method for creating a multiple, tuned optical grating within the filtering member of the exemplary information analyzer; and

FIG. 6 illustrates an exemplary method for manufacturing a compact and portable biosensing device.

### DETAILED DESCRIPTION

The detailed description set forth below is intended as a description of exemplary embodiments and is not intended to represent the only embodiments in which the present biochemical sensing devices and methods can be practiced. The term “exemplary” used throughout this description means “serving as an example, instance, or illustration,” and should not necessarily be construed as preferred or advantageous over other embodiments. The detailed description includes specific details for the purpose of providing a thorough understanding of the biochemical sensing devices and methods. However, it will be apparent to those skilled in the art that the biochemical sensing devices and methods may be practiced without these specific details. In some instances, well-known structures and devices are shown in block diagram form in order to avoid obscuring the concepts of the biochemical sensing devices and methods.

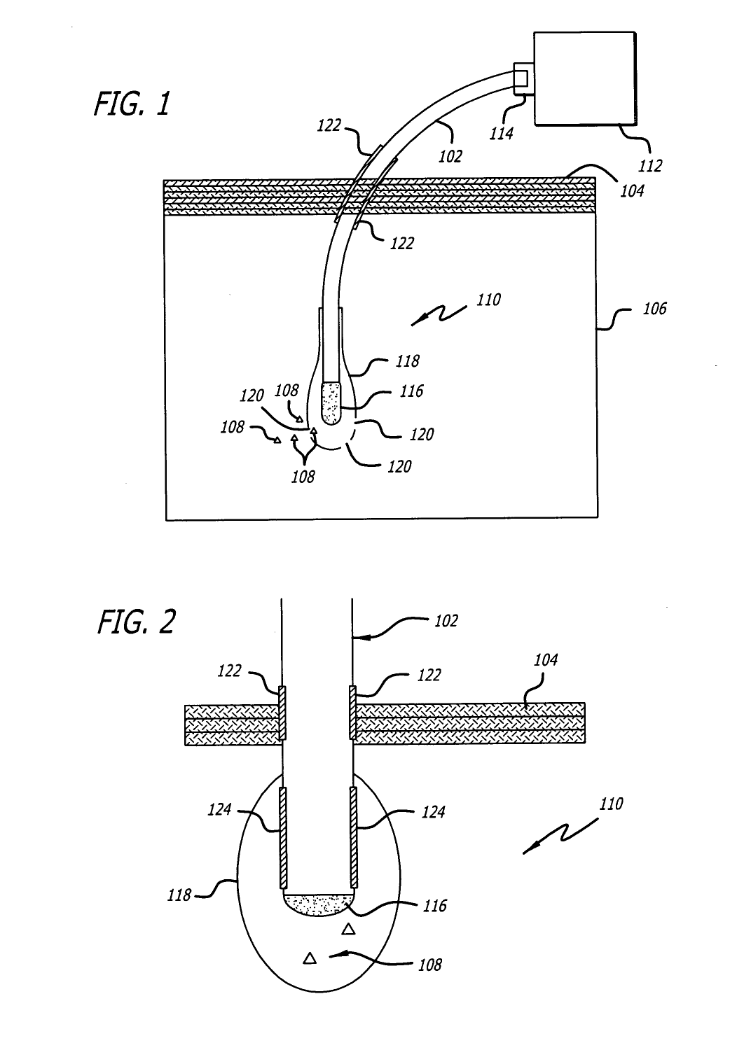

FIG. 1 illustrates a compact and portable biosensing device and an exemplary mode of positioning relative to a patient's body. The exemplary biosensing device comprises a transmitting member 102 that extends through the patient's skin 104. The transmitting member 102 may be injected in a percutaneous region of the patient's body 106, or in any other region in which analytes 108 are being tested. The biosensing device includes a biosensor element 110, attached to the end of the transmitting member 102 that is inserted into the patient's body. The opposite end of the transmitting member 102 may be attached to an information analyzer 112, by means of a connector 114. The information analyzer 112 receives information directed from the biosensing element 110 through the transmitting member 102, then filters and analyzes the received information to detect the presence or quantity of analytes within the patient's body 106.

In an exemplary embodiment, the transmitting member 102 is a single optical fiber. It is to be understood that the transmitting member might include additionally other channels required to initiate or modify the sensing function. For example, a second optical fiber might be incorporated to provide photonic excitation of a chemical or fluorescence reaction. In another example, a fine electrical wire might be incorporated to apply an electrical current or biasing voltage or to generate electrolysis of the body fluids to induce pH changes. In yet another example, a loop of electrical wire might be incorporated as a heating element. In yet another example, a capillary tube might be incorporated to allow introduction of a chemical enzyme or initiator of the reaction to be detected via an optical fiber in the transmitting member.

In an exemplary embodiment, the information transmitted through the optical fiber 102 is light energy (photons at different wavelengths), and the connector 114 is an optical connector, to ensure the presence of an optical connection between the optical fiber 102 and the information analyzer 112. In this embodiment, the information analyzer 112 exposes the biosensor element 110 to excitation light of a first wavelength that is directed through the optical fiber 102 to the biosensor element, and in response receives emitted fluorescent light of at least a second wavelength from the biosensor element 110, directed through the optical fiber 102 in the opposite direction. The emitted fluorescent light is then filtered and analyzed by the information analyzer 112 to identify and/or quantify the analytes detected by the biosensor element 110. The information analyzer 112 may identify the presence of specific analytes by detecting the specific wavelength of the fluorescent light emitted, and measures the quantity of analytes present by measuring the intensity of the fluorescent light emitted. The directing of these various forms of light, and the general configuration of the biosensor element 110, are now described in further detail.

The biosensor element 110 comprises biosensing material 116 located substantially at the end of the optical fiber 102. In a first exemplary embodiment, the biosensing material 116 may be attached directly to the internal end of the optical fiber 102. In an alternative exemplary embodiment, the biosensing material 116 may be inserted into the patient's body 106 separately from the optical fiber 102, and the optical fiber 102 positioned in proximity to the implanted biosensing material 116. In another exemplary embodiment, it may be desirable to prevent substantially direct contact between the biosensing material 116 and patient tissue 106. In this case, the biosensor element 110 includes a containment matrix 118 that substantially contains the biosensing material 116 within a reaction region that is in close proximity to the end of the optical fiber 102. Further, the containment matrix 118 may be configured to be in contact with or form a seal with the optical fiber 102. The containment matrix 118 thereby can contain the biosensing material so that it does not diffuse away from the biosensor element. The containment matrix 118 might also contain the products of a reaction between analytes 108 and the biosensing material 116. This containment of the reactive products can prevent them from dispersing throughout the patient's body such that they are retained within a concentrated area for signal communication to the optical fiber 102. The containment matrix 118 can include pores 120 to allow analytes 108 to diffuse within the containment matrix 118 to contact the biosensing material. The pores 120 may be inherently formed due to the characteristics of the material used for the containment matrix or, if the selected material is not sufficiently porous, then pores may be explicitly created therein, for example by burning holes using a tightly focused laser beam such as an excimer laser. The pores are sized such that they are large enough to allow the diffusion of analytes 108 into the reaction region, and small enough to prohibit the passage of other elements from the reactive region to other areas of the patient's body. Alternative preferred embodiments do not possess the containment matrix 118.

FIG. 2 illustrates an exemplary biosensor element 110 and certain other features of the exemplary biosensing device. The optical fiber 102 may be composed of a number of different materials such as, for example, glass, silicon or plastic. While different materials may be used in any of the embodiments described herein, glass has desirable optical properties and can be configured to have a silicon outer surface that can be modified to bind different coatings (discussed below). Although the optical fiber 102 does not have a specific size requirement, fibers having a diameter between about 50 μm and about 200 μm are used for ease of insertion through the skin 104 of a patient. Fibers within this range of sizes are also sufficiently large for effective data transmission, suitably flexible that a patient can manipulate them with ease, and sufficiently strong to withstand patient wear. For example, a 110 μm glass fiber with a polyimide sheath can be bent to a radius of about 2 mm before fracturing.

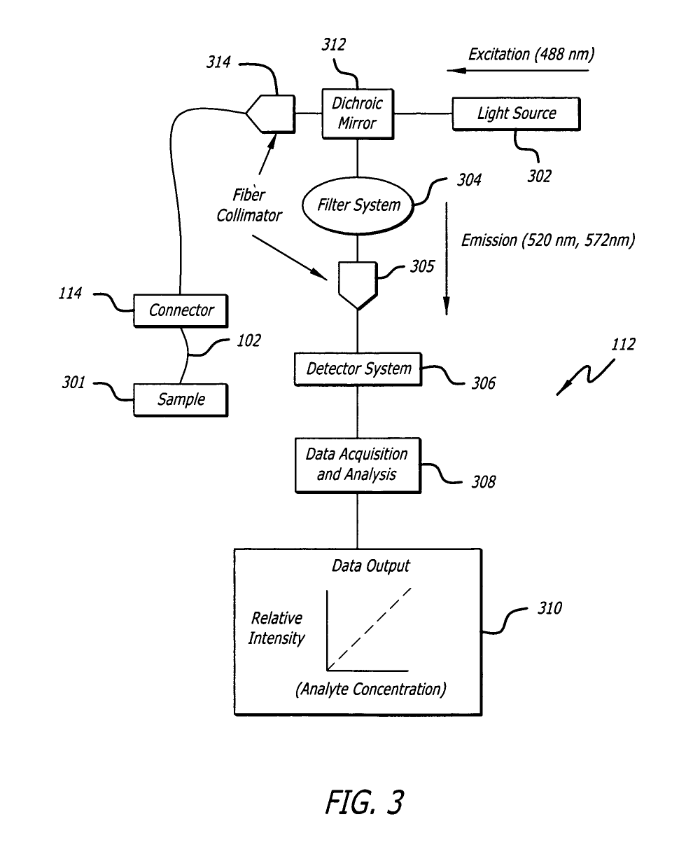

FIG. 3 is a functional block diagram of an exemplary information analyzer. In the exemplary embodiment, the information analyzer 112 is a photonic analyzer. Specifically, the information analyzer is a fluorescence spectrophotometer that photonically excites a sample 301 within, or in proximity to the biosensor element 110, and then detects the wavelength and/or intensity of any optical signal emitted therefrom. The information analyzer 112 includes a light source 302, one or more optical filters 304, a photon detector 306, signal processing electronics 308 and a patient readout system 310.

In an exemplary method employed by the information analyzer 112, an excitation wavelength is produced by light source 302. The light source 302 may be, for example, a fiber-coupled blue laser diode with a built-in source driver capable of producing, for example, 20 mW-24 mW. Alternatively, blue light-emitting diodes (LED) with high output power may be used as the light source 302. Those skilled in the art will also recognize other suitable excitation light sources such as a broadband, incandescent light source from which a tunable, narrow band of excitation wavelengths can be selected by a diffraction grating or prism.

In an exemplary embodiment, an excitation wavelength is produced by light source 302. Of course, a wide variety of excitation wavelengths and fluorescence changes may be used in methods of the invention, according to the sample 301 being tested (for example, W. P. Van Antwerp and J. J. Mastrototaro, U.S. Pat. No. 6,319,540, Nov. 20, 2001, herein incorporated by reference). In alternative embodiments, a detectible wavelength of light is produced as a result of various reactions between analytes and the biosensing material 116, rather than by excitation light.

The excitation light passes through a mounted dichroic mirror 312 and through a first fiber collimator 314, where it is focused. It then passes through optical connector 114, such as an AMP connector, which may be attached to the external end of the optical fiber 102 element of the biosensing device when a measurement is to be made. The light continues through the optical fiber 102 and to the internal end of the optical fiber 102. Upon excitation of any fluorophores present in the biosensing material, fluorescent wavelengths are emitted.

The fluorescent emissions are directed back through the optical fiber 102 and connector 114 to the information analyzer 112. Where a single fiber is used, the fluorescent emission travels via the internal end of optical fiber 102 into the connector 114 and through the first fiber collimator 314 of the information analyzer. The fluorescent emission can be deflected (for example 90°) by the dichroic mirror 312 into a filter system 304. For the exemplary process described below, in which two emission wavelengths are produced, the filter system could be, for example, a two-wavelength interference filter system that is mounted on a motorized, time-controlled filter wheel. The filter wheel mechanically alternates two interference filters to produce two narrowband signals centered at the emitted wavelengths. The narrowband signals are then focused at a second fiber collimator 305 and measured with a photo multiplier tube (PMT) detection system 306. For example, during a first interval, the excitation light intensity will produce a corresponding fluorescence emission intensity that will be measured by detector system 306. During a second interval, the same excitation light intensity will produce a corresponding fluorescence emission intensity that will be measured by detector system 306.

This cycle can be repeated numerous times when testing for analytes, and processed as described below. Alternatively, filter system 304 may employ optical grating filters within the filtering member 408.

Continuing with the description of FIG. 3, signal processing electronics 308 may, for example, compute the average and standard deviation for each sampling interval. The ratio (relative intensity) of the emission intensity of fluorescence is then calculated and plotted as a function of analyte concentration. Results are displayed to the patient via a patient readout system 310 that may be, for example, an LED display, a numeric liquid crystal display or computer display located on a handheld information analyzer 112. The information can be displayed to the patient in various formats including, for example, alphanumeric readouts, on-screen icons or symbols, or various forms of graphs or charts.

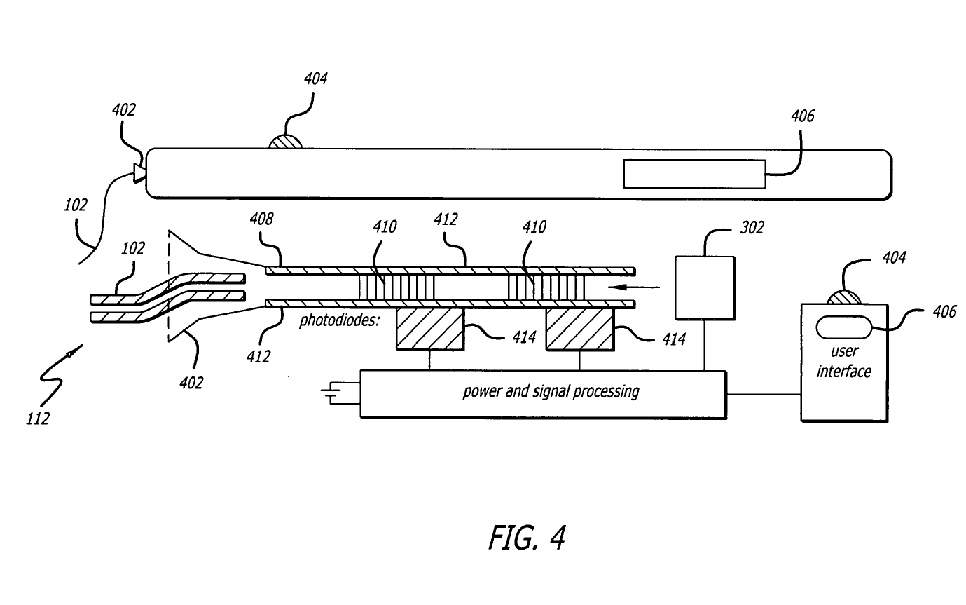

FIG. 4 illustrates an exemplary information analyzer 112 configuration that is sized and configured to be easily carried by the patient. The information analyzer 112 is portable such that it may be easily moved or even worn by the patient sized and configured to be easily carried by the patient. For example, the information analyzer 112 could be sized to fit within a patient's hand, and could be light enough to be easily moved by the patient, or attached to the patient's clothing or to a strap that is worn by the patient. Because of its portability and small size, the information analyzer 112 may be used to take continuous measurements, such as when the patient wears it on his body or clothing. Its small size also makes the information analyzer 112 convenient for taking frequent, yet intermittent measurements, such as when the patient wears it or simply carries it with him because it is easily portable and accessible. In use, the patient slips the free external end of the optical fiber 102 of the implanted biosensing device into a connector 114, triggers a reading with a button 404 and views the results on a display 406. The conical orifice 402, in an exemplary embodiment, is a self-centering optical fiber connector, causing optical fiber 102 to be optically aligned with the filtering member 408 of the information analyzer 112.

As described in U.S. Pat. No. 6,058,226 (Starodubov), which is incorporated herein by reference, the filtering member 408, (which may also be an optical fiber), includes a tunable filter grating region 410 and a light source 302 such as a laser diode at one end. As fluorescent emissions from the fluorophore pass into the filter section 410, they are detected by detectors 414. In an exemplary embodiment, the filtering member 408 may also include a cladding layer 412, which could be, for example, a material similar in nature to the core of the optical fiber but with a different index of refraction. The cladding layer 412 is used to capture light that is deflected into it by the filtering member 408. Gratings in optical density within the filtering member 408 deflect photons with wavelengths matched to the grating wavelength into the cladding layer 412. Wavelengths traveling within the cladding layer 412 are captured and quantified by detectors 414 which may be, for example, photodiodes. Signal processing electronics 308 then receive information from detectors 414, analyze the information, and provide details to the patient by presenting them on display 406.

In manufacturing components for the information analyzer 112, the filtering member, which in the exemplary embodiment is optic fiber 408, is modified so that it contains alternating regions of higher and lower optical density at longitudinal intervals. These alternating regions of varying density create a wavelength specific optical grating within the filtering member 408. When light of a specific wavelength passes through that region of the fiber, it is deflected laterally into the cladding layer 412. This deflected light tends to continue propagating in the same longitudinal direction within the cladding, but it is highly deflected in the thin cladding layer and easily captured with a detector 414, such as a high efficiency photodiode, coupled to the side of the filtering member 408. Other wavelengths of light traveling through a given filter region of the filtering member 408 that is not tuned to those wavelengths pass without significant absorption or deflection. This makes it possible to design several adjacent optical gratings within the filtering member 408, each tuned to a different wavelength, to act as a series of high-Q spectral filters. Thus, multiple analytes can be detected using an information analyzer configured to selectively filter multiple wavelengths of emitted light from one another.

The tendency of light that is deflected into the cladding layer to have a weak longitudinal propagation is also advantageous when dealing with strong excitation light traveling in a direction opposite to the weak emission light to be detected. As will be recognized by those skilled in the art, carefully spacing the filter regions and photodiodes will minimize the detection of any excitation light that may be deflected into the cladding due to non-ideal properties of the filters.

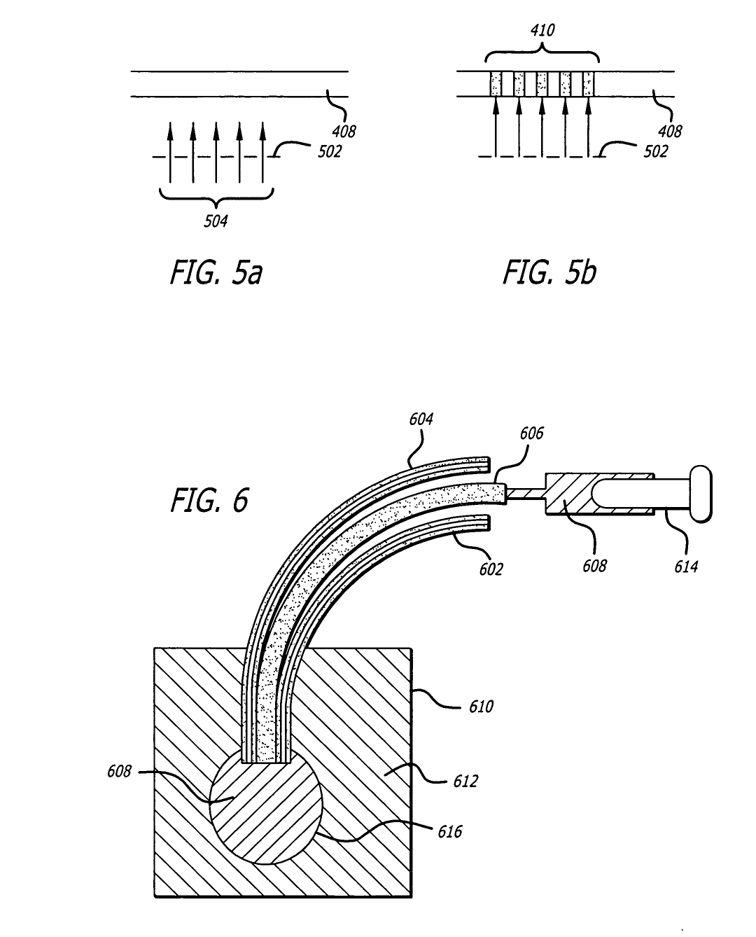

FIGS. 5a and 5b illustrate an exemplary method for creating multiple tuned optical gratings within the filtering member 408 of the information analyzer 112. First, the filtering member 408 is made photo-sensitive by replacing some of the material with germanium. For example, in an embodiment having a silicon optical fiber 408, a portion of the silicon is replaced with germanium, which has photosensitive properties. Then, in FIG. 5a, an optical pattern 502 having the desired grating spacing is created by diffraction, and used to illuminate the fiber 408 from the side as indicated by arrows 504. The incident photons are absorbed by the germanium, precipitating a condensation in the local crystal lattice that changes its optical density, as illustrated in FIG. 5b. The pattern of this condensation becomes an optical grating region 410 within the filtering member 408. By this procedure, the filtering member 408 can be “photonically programmed” by brief exposure to patterned light to act as a custom multi-channel optical filter as required.

In use, to avoid damage to the optical fiber 102 of the implanted biosensing device during daily activities of the patient such as bathing and grooming, the external end of the fiber 102 in one embodiment is unencumbered by extraneous mechanical features. For example, connector 114 is attached to information analyzer 112 rather than to optical fiber 102. Optical fiber 102 is inserted into connector 114 only when a reading is to be taken. In one embodiment the connector 114 can be self-centering, such that optical fiber 102 is coaxially aligned with a mating optical fiber within information analyzer 112 in order to achieve adequate optical coupling. As shown in FIG. 4, this can be accomplished by shaping the orifice of connector 114 as a gradually tapering, truncated cone whose apical plane is formed by filtering member 408 that is made from an optical fiber having the same diameter as optical fiber 102. Also, some embodiments may involve the patient dipping the exposed end of the implanted optical fiber 102 into a combination cleaning and optical coupling solution before inserting it into the connector 114, to provide a fluid bridge between any physical gap that might remain between the transmitting and filtering members within the connector 114. Alternatively, connector 114 could be irrigated with a cleaning and optical coupling solution before optical fiber 102 is inserted into it. The optical coupling must be sufficient for the efficient transmission of excitation light directed toward biosensor element 110 and emission light received from biosensor element 110.

The various embodiments described herein may be constructed with a variety of different components and materials. For example, while various exemplary embodiments described above comprise a single optical fiber for transmitting light in two directions, it is also possible that a biosensing device or an information analyzer may utilize two separate transmitting members, each one propagating information in a single, different direction.

Multiple fluorescent labels can be used on the same sample and individually detected quantitatively, permitting measurement of multiple cellular responses simultaneously. Many quantitative techniques have been developed to harness the unique properties of fluorescence including: direct fluorescence measurements, fluorescence resonance energy transfer (FRET), fluorescence polarization or anisotropy (FP), time resolved fluorescence (TRF), fluorescence lifetime measurements (FLM), fluorescence correlation spectroscopy (FCS), and fluorescence photobleaching recovery (FPR) (Handbook of Fluorescent Probes and Research Chemicals, Seventh Edition, Molecular Probes, Eugene Oreg.).

In exemplary embodiments, quantum dots may be used as fluorescing indicators. Highly luminescent semiconductor quantum dots (zinc sulfide-capped cadmium selenide) have been covalently coupled to biomolecules for use in ultrasensitive biological detection (Stupp et al. (1997) Science 277(5330):1242-8; Chan et al. (1998) Science 281(5385):2016-8). Compared with conventional fluorophores, quantum dot nanocrystals have a narrow, tunable, symmetric emission spectrum and are photochemically stable (Bonadeo et al. (1998) Science 282(5393):1473-6). The advantage of quantum dots is the potential for exponentially large numbers of independent readouts from a single source or sample. In addition, water-soluble gold-based quantum dots having favorable fluorescence characteristics can be used (gold quantum dots are described in Zheng J, Zhang C, Dickson R, “Highly fluorescent, water-soluble, size-tunable gold quantum dots,” Phys Rev Lett. Aug. 13, 2004;93(7):077402, which is hereby incorporated by reference).

Alternative embodiments may utilize immunoassay techniques as radioimmunoassay (RIA) or enzyme linked immunosorbance assay (ELISA), homogeneous enzyme immunoassays, and related non-enzymatic techniques. These techniques utilize specific antibodies as reporter molecules, which are particularly useful due to their high degree of specificity for attaching to a single molecular target.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device; discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

In an exemplary embodiment, the biosensing device detects the presence of analytes within the patient's tissues by employing a biosensing material 116. A chemical binding or reaction between the analyte 108 and the biosensing material 116 gives rise to a state change that can be transmitted to and detected by the information analyzer 112. The biosensing material 116 takes advantage of the unique specificity of biosensing molecules for analyte(s) of interest. This high selectivity allows the analyte to be measured even when mixed with other substances, such as occurs in blood, tumors, or extracellular fluids. The biosensor materials can be selected to maintain mechanical stability and biocompatability during chronic implantation.

In an exemplary embodiment, the biosensing material 116 comprises an analyte-specific biomolecule immobilized in a polymer matrix which is in contact with the internal end of the transmitting member. In this embodiment, the biosensing material is presented at least on the surface of the polymer matrix, such that when analytes 108 diffuse into the region having the biosensing material 116, analytes 108 selectively bind with the biosensing material. The interaction of the analyte with the biosensing material results in a detectable state change. (i.e., changed fluorescent properties of the analyte, biosensing materials or reaction products.)

Receptors, antibodies, enzymes that specifically interact with the analyte(s) to be detected, and biomolecules associated with the presence and/or destruction of cancer cells may be immobilized by physical capture within or covalent bonding to a biocompatible, polymeric matrix such as can be formed by the polymerization of various analogues of ethylene oxides to form, for example, polyethylene glycol. In one alternative embodiment, the bibsensing material 116 may be fluidic or cellular in nature requiring encapsulation in a containment matrix (described below).

In another alternative embodiment, the biosensing material may include whole cells which are selected for or modified to respond to the selected analyte to produce a state change which is transmitted to and detected by the analyzer. For example, cells may be harvested from the patient (ex. endothelial cells) and modified for use as a biosensing material 116 by genetic engineering to cause them to express, for example, particular receptor molecules for the analyte 108 to be measured. Where the cells naturally express these molecules that interact selectively with the analyte 108 to be detected, these cells may be induced to take up fluorescent dyes that bind to these molecules and that change the intensity or wavelength of their fluorescence depending on the state of interaction between the molecules and the analyte 108.

Modification of cells may include transfecting the cells to express molecules which specifically react with the analyte of interest, as described, for example, in Tom Maniatis, “Molecular Cloning: a Laboratory Manual,” 3rd Ed. For example, cells may be transfected with expression vectors encoding a receptor which selectively binds to the selected analyte. The receptor may be one located in the cell membrane (such as a transmembrane receptor), bound to the cell membrane (internal or external) or intracellular (e.g., lipid soluble analytes such as steroids).

Binding of the analyte to the receptor may then trigger a state change within the cells detectable by the information analyzer. For example, binding of an analyte (ex. hormone) to an expressed intracellular receptor may act as a transcription factor to enhance the expression of a second construct having a reporter (ex. luciferase) having fluorescent properties.

Modification of whole cells may also include dye loading. For example where the analyte selectively binds to a transmembrane receptor, the cell undergoes a sequence of chemical changes (i.e., ion fluxes) resulting in state changes which are detectable. For example, the changes could include opening of calcium channels through the cell membrane, increasing intracellular calcium, and the change in the fluorescence of a calcium sensitive dye (such as FURO-2) that has previously been loaded into cells.

As indicated above, biosensing devices may employ several control and modulation methods: electrochemical, optical, thermal and mechanical. Turner A, Karube I and Wilson G. (1987) Biosensors: Fundamentals and Applications. London: Oxford Science Publications; Hall E. (1991) Biosensors. New Jersey: Prentice Hall; Fraser, D. (1997) Biosensors in the body: continuous in vivo monitoring. Chichester, N.Y.: Wiley; Blum, L. (1991) Biosensor Principles and Applications. New York: M. Dekker; Buck, R P. (1990) Biosensor Technology: Fundamentals and Applications. New York: M. Dekker, herein incorporated by reference.

In an exemplary embodiment fluorescence optical sensing is utilized. Here, the biosensing material includes molecules that undergo a change in fluorescent emission in proportion to the concentration of analyte of interest in the surrounding medium. For use in this system, many different fluorescent dyes have been developed and these can be bound covalently to molecules that bind specifically to analytes. Thompson, R. B. “Fluorescence-Based Fiber-Optic Sensors.” Topics in Fluorescence Spectroscopy, Vol. 2: Principles. New York: Plenum Press 1991: 345-65; McNichols R and Cote G. “Optical glucose sensing in biological fluids: an overview.” Journal of Biomedical Optics January 2000, 5:5-16; Czarnik, A. (1993) Fluorescent Chemosensors for Ion and Molecule Recognition. Washington: American Chemical Society, herein incorporated by reference. Other optical sensing techniques may be used such as absorption and transmission which are well known to individuals skilled in the art.

Two particular embodiments can be useful where fluorescence is selected as the mode of optical transmission: 1) the analyte is itself fluorescent; or 2) the analyte is not fluorescent but interacts with a fluorophore that emits a fluorescent signal. Krohn, D. (1988) Fiber Optic Sensors: Fundamentals and Applications. North Carolina: Instrument Society of America. Where the analyte to be detected is glucose, a number of techniques may be employed, including, but not limited to enzyme based and competitive affinity binding. McNichols R and Cote G. “Optical glucose sensing in biological fluids: an overview.” Journal of Biomedical Optics January 2000, 5:5-16.

Many foreign molecules such as pharmacological agents can be detected by a fluorescence method that is based on affinity binding (immunoassay) between an antibody and an antigen. The antibody can be considered the molecular-recognition element (biosensing molecule), which binds reversibly with a specific antigen or analyte. Monoclonal antibodies can be useful because they provide a relatively pure source of a single antibody with a high affinity for a specific antigen. Monoclonal antibodies can be coupled to fluorescent dyes such as TRITC and FITC in such a way as to produce fluorescence whose intensity or wavelength is modulated depending on whether the antibody is bound to the antigen or not.

A containment matrix 120 may be useful in some embodiments. As mentioned, the materials of the containment matrix 120 can be selected to be biocompatible with the patient, permeable to the analytes being detected, minimally permeable to the reporting molecule being detected (i.e., fluorophores) and of a material which preferably forms a strong adhesion to the transmitting member in the absence or presence of additional adhesion coatings (described above). The containment matrix can be attached directly to the internal end of the transmitting member, permitting efficient and constant coupling to a small sensing structure. In an exemplary embodiment, polyethylene glycol (PEG) polymers are used as PEG demonstrates good biocompatability and structural integrity. The polymer can be applied to the transmitting member in an unpolymerized state, then polymerized to enhance stability of the structure by gamma irradiation, chemical cross-linking or UV radiation. One exemplary formula is described below.

In some cases, it may be possible to transmit both the exciting and emitted light over the same fiber, for example, if a brief excitation pulse of light produces a longer-lived fluorescence that an be detected after the excitation pulse has been extinguished. In other cases it may be necessary to have two optical fibers 102, one of which is used to transmit the excitation light inward while the outgoing fluorescent response is transmitted to a separate detector by the other. FIG. 6 illustrates an exemplary method for manufacturing a compact and portable biosensing device having a biosensing member surrounded by a containment matrix. The biosensing device could have, for example, at least two separate optical fibers 602 and 604 bundled together.

In some cases such as when the biosensing element 110 includes living cells, it may be necessary to retain the biosensing element 110 as a fluid on the internal end of the transmitting member 102. In the exemplary method depicted in FIG. 6, the transmitting member 102 contains a capillary tube 606. A suspension of biosensing material 608 may be prepared for injecting through the external end of the capillary tube 606 within an injector 614. The internal end of the assembly may be placed in a vacuum chamber 610 and exposed to vapor 612 of a vapor-depositable material. An exemplary polymer that could be used is Parylene-C (Registered trademark of Union Carbide Corp. for poly-monochloroparaxylylene), however those skilled in the art will recognize that a number of vapor-depositable material vapors may be employed. As the vapor 612 condenses and polymerizes on the surface of the bundle, a droplet of biosensing material 608 is injected through capillary tube 606 by injector 614, which can be, for example, a common hypodermic needle. The droplet cools rapidly by evaporation in the vacuum chamber 610. The low surface temperature of the rapidly cooling droplet causes a high rate of vapor condensation and polymerization, which forms containment matrix 616 around the droplet. Containment matrix 616 adheres to the optical fiber 602, forming a seal that retains biosensing material 608. If the analyte does not diffuse through the containment matrix 616, pores may be created through containment matrix 616 by various means such as laser ablation.

Other materials for the containment matrix are available, such as lipid or other semi permeable membranes that might not require the formation of pores therein. In an exemplary embodiment, membranes with special properties could be layered over the biosensing material 116, for example semipermeable or ion selective membranes may be used to form a containment matrix 118. In this example, the ratio of the cross-sectional area of the pore 120 to the volume of the biosensing material 116 may function as a preset control on the rate at which the analyte enters the sensing area through the containment matrix 118. This particular embodiment may be useful for quantitative assays of small molecules by non-cellular biosensor materials such as enzymes or antibodies, e.g., glucose sensor for diabetics. See for example, U.S. Pat. No. 6,063,637 to Arnold et al., herein incorporated by reference.

It will be recognized that alternative embodiments of the biosensing device may require alternative manufacturing processes. For example, in an exemplary embodiment involving cells as the biosensing material 508, the cells may be grown onto the tip of an optical fiber 502 in tissue culture. When the tip of the optical fiber 502 is removed from the aqueous culture medium, a droplet of cells and media cling to the tip and may then be coated with condensed vapor 512 according to a cooling and condensing method as described above.

In some embodiments the biosensing material of the invention is designed for implantation subcutaneously into a well-vascularized space (such as the scalp). However, it should be noted that intravascular and implantation within an organ may be desirable for measuring some analytes. In other embodiments, implantation into tumors is desired for detecting specific analytes and/or the effectiveness of various therapeutic agents to treat cancer patients. The transmitting member can also be implanted at least partially subcutaneously for local detection (such as photonic detection) from the biosensing material. The biosensing materials and transmitting member can be constructed so as to remain stably positioned within a patient's body for repeated measures of analyte for a selected time, such as at least one to three months or longer. However, the biosensor and transmitting member are also constructed and implanted so as to be wholly and completely removable from the patient when the biosensor is no longer functioning or required. Alternatively, the biosensing element 110 may be constructed so as to detach from the transmitting member during its removal and to biodegrade in situ. The close coupling afforded between the optical fiber and the biosensor permit the volume of the biosensor to be very small, reducing the possibility of a toxic or immunogenic reaction to the biosensing element 110 as it degrades.

In an exemplary embodiment such as shown in FIG. 1 or 2, the outer surface of the transmitting member 102 may be modified such that various coatings may be applied to regions of the outer surface of the member. Coatings which improve biocompatibility with patient tissues generally and integration with the skin are desirable so that a long-lived stable interface is formed between the transmitting member and the body. Any selected portion of the transmitting member 102 (such as the region of the transmitting member 122 passing through the skin 104) or the entire length of the fiber may be coated with substances which promote cellular attachment. For example, the transmitting member may be coated with a naturally occurring protein, collagen to encourage a stable percutaneous interface (U.S. Pat. No. 5,800,545 to Yamada et al., Sep. 1, 1998, incorporated herein by reference). Example 1 below describes the surface modification of a glass optical fiber by acylation in an acrylol chloride solution, thereby providing a covalent binding site for attachment of the acrylate group of the polyethylene glycol. This and similar adhesion promoters can be used to attach firmly coatings to the optical fiber that provide stable, biocompatible attachment to the skin. Such coatings may be prepared in layers, such as an inner covalent binding site on the optical fiber, a hydrogel polymer such as polyethylene glycol bound to the binding sites, and a layer of collagen or other naturally occurring protein attached to the polymer that becomes integrated into the surrounding tissues of the skin.

Further, coatings could be applied to the outer surface of the transmitting member 102 to enhance the attachment of the biosensing material 116 and/or the containment matrix 118 thereto. These coatings may facilitate a stable interface between the transmitting member and the biosensing material such that these components of the device remain in operative communication. Further, these coatings may encourage a stable interface between the transmitting member and the containment matrix, so that where desired, the biosensing material is isolated from the patient tissue and reaction products are maintained in a concentrated area for detection. Finally, a stable interface may be desirable such that when the device is removed from the patient it can be removed as a single structure. As shown in the exemplary embodiment of FIG. 2, the transmitting member internal end 124 may be coated for these purposes. Where containment matrix 118 is formed of substantially polyethylene glycol (PEG), a silicon outer surface of the transmitting member 102 may be prepared with a commercial trichlorosilane as an adhesion promoter. Where a plastic transmitting member 102 is used, their typical Teflon outer jacket provides a surface that binds tightly to PEG derivatives that have been end-functionalized with 1-4 perfluorocarbon groups that have been shown to strongly adsorb physically to Teflon surfaces (Hogen-Esch, T. E.; Zhang, H.; Xie, X. “Synthesis and Characterization of Well Defined End-Functionalized Hydrocarbon and Perfluorocarbon Derivatives of Polyethyleneglycol and Poly(N.N-dimethylacrylamide), Chapter 11, in “Associative Polymers in Aqueous Solutions,” ACS Symposium Series Vol. 765, pp. 179-203, J. E. Glass Editor, 2000). Covalent binding can also be obtained to either surface by glow-discharge pretreatment of the fiber surface. Wang, P. Tan, K. L. and Kang. E. T. “Surface modification of poly(tetrafluoroethylene) films via grating of poly(ethylene glycol) for reduction in protein adsorption.” Journal of Biomedical Science and Polymer Edition. 2000, 11:169-186. A glass optical fiber 102 can be pretreated with Ar plasma before graft copolymerization and then exposed to the atmosphere for about 10 min to effect the formation of the surface peroxides and hydroperoxides. The peroxide will form a covalent bonding with the acrylate group of PEG.

Embodiments of the biochemical sensing devices and methods can be adapted to detect and/or analyze the effectiveness of chemotherapy agents on cancer and/or normal cells. Most chemotherapeutic agents fall into the following categories: alkylating agents, antimetabolites, antitumor antibiotics, corticosteroid hormones, mitotic inhibitors, and nitrosoureas, hormone agents, miscellaneous agents, and any analog or derivative variant thereof. Chemotherapeutic agents and methods of administration, dosages, etc. are well known to those of skill in the art (see for example, the “Physicians Desk Reference”, Goodman & Gilman's “The Pharmacological Basis of Therapeutics” and in “Remington's Pharmaceutical Sciences”, incorporated herein by reference in relevant parts).

Agents or factors suitable for analysis may include any chemical compound that induces DNA damage when applied to a cell. Chemotherapeutic agents include, but are not limited to, 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP16), famesyl-protein transferase inhibitors, gemcitabine, ifosfamide, mechlorethamine, melphalan, mitomycin, navelbine, nitrosurea, plicomycin, procarbazine, raloxifene, tamoxifen, taxol, temazolomide (an aqueous form of DTIC), transplatinum, vinblastine and methotrexate, vincristine, or any analog or derivative variant of the foregoing.

An exemplary embodiment can be adapted to detect and/or analyze the effectiveness of alkylating agents that directly interact with genomic DNA to prevent the cancer cell from proliferating. Embodiments can be used to detect the effectiveness of chemotherapeutic alkylating agents that affect all phases of the cell cycle. Alkylating agent that can be analyzed may include, but are not limited to, a nitrogen mustard, an ethylenimene, a methylmelamine, an alkyl sulfonate, a nitrosourea or a triazines. They include but are not limited to: busulfan, chlorambucil, cisplatin, cyclophosphamide (cytoxan), dacarbazine, ifosfamide, mechlorethamine (mustargen), and melphalan.

Another exemplary embodiment can be adapted to detect and/or analyze the effectiveness of chemotherapeutic antimetabolites that disrupt DNA and RNA synthesis. Various categories of antimetabolites that may be analyzed include, but are hot limited to, folic acid analogs, pyrimidine analogs and purine analogs and related inhibitory compounds. Specific antimetabolites that may be analyzed include but are not limited to, 5-fluorouracil (5-FU), cytarabine (Ara-C), fludarabine, gemcitabine, and methotrexate.

Other exemplary embodiments can be adapted to detect and/or analyze chemotherapeutic agents originally isolated from a natural source. Such compounds, analogs and derivatives thereof may be isolated from a natural source, chemically synthesized or recombinantly produced by any technique known to those of skill in the art. Natural products to be analyzed include but are not limited to such categories as mitotic inhibitors, antitumor antibiotics, enzymes and biological response modifiers.

Further exemplary embodiments can be adapted to detect and/or analyze mitotic inhibitors such as plant alkaloids and other natural agents that can inhibit either protein synthesis required for cell division or mitosis. Mitotic inhibitors that may be analyzed include but are not limited to, for example, docetaxel, etoposide (VP16), teniposide, paclitaxel, taxol, vinblastine, vincristine, and vinorelbine. Taxoids, which are a class of related compounds isolated from the bark of the ash tree, Taxus brevifolia, can also be analyzed by embodiments of the present invention. Taxoids include but are not limited to compounds such as docetaxel and paclitaxel. Furthermore, embodiments can be adapted to analyze the effectiveness of vinca alkaloids, including but not limited to compounds such as vinblastine (VLB) and vincristine.

Another exemplary embodiment can be adapted to detect and/or analyze the effectiveness of antitumor antibiotics that interfere with DNA by chemically inhibiting enzymes and mitosis or altering cellular membranes. Examples of antitumor antibiotics that can be analyzed by such embodiments include but are not limited to, bleomycin, dactinomycin, daunorubicin, doxorubicin (Adriamycin), plicamycin (mithramycin) and idarubicin.

Further exemplary embodiments can be adapted to detect and/or analyze the effectiveness of hormones used to kill or slow the growth of cancer cells. For example, corticosteroid hormones, such as prednisone and dexamethasone, may be detected and/or analyzed by preferred embodiments. Futhermore, embodiments may be adapted to analyze the effectiveness of: progestins (such as hydroxyprogesterone caproate, medroxyprogesterone acetate, and megestrol acetate); estrogens (such as diethylstilbestrol and ethinyl estradio); antiestrogens (such as tamoxifen); androgens (such as testosterone propionate and fluoxymesterone); antiandrogens (such as flutamide); and gonadotropin-releasing hormone analogs (such as leuprolide).

Additional chemotherapeutic agents that may be detected and/or analyzed by embodiments include, but are not limited to: platinum coordination complexes, anthracenedione, substituted urea, methyl hydrazine derivative, adrenalcortical suppressant, amsacrine, L-asparaginase, and tretinoin, can also be analyzed alternative preferred embodiments. Futhermore, embodiments may also analyze the effectiveness of anti-angiogenic agents including but not limited to angiotensin, laminin peptides, fibronectin peptides, plasminogen activator inhibitors, tissue metalloproteinase inhibitors, interferons, interleukin 12, platelet factor 4, IP-10, Gro-β, thrombospondin, 2-methoxyoestradiol, proliferin-related protein, carboxiamidotriazole, CM101, Marimastat, pentosan polysulphate, angiopoietin 2 (Regeneron), interferon-alpha, herbimycin A, PNU145156E, 16K prolactin fragment, Linomide, thalidomide, pentoxifylline, genistein, TNP-470, endostatin, paclitaxel, accutin, angiostatin, cidofovir, vincristine, bleomycin, AGM-1470, platelet factor 4, and minocycline.

Embodiments can also be adapted to detect and/or analyze various biomolecules associated with cellular metabolism and/or structure, cancer and/or effective treatment of cancer cells. Such biomolecules include but are not limited to: lipids, carbohydrates, organic or inorganic molecules, nucleic acids, proteins, metabolites, functional states of proteins, enzymes, cytokines, chemokines, and other factors, e.g. growth factors, such factors include GM-CSF, G-CSF, M-CSF, TGF, FGF, EGF, TNF-α, GH, corticotropin, melanotropin, ACTH, extracellular matrix components, surface membrane proteins, such as integrins and adhesins, soluble or immobilized recombinant or purified receptors, and antibodies against receptors or ligand mimetics.

Further embodiments can detect and/or analyze other parameters of interest, including detection of cytoplasmic, cell surface or secreted biomolecules, frequently biopolymers, such as polypeptides, polysaccharides, polynucleotides, and lipids. Cell surface and secreted molecules are a preferred parameter type as these mediate cell communication and cell effector responses and can be more readily assayed. In some embodiments, parameters include specific epitopes. Epitopes are frequently identified using specific monoclonal antibodies or receptor probes. In some cases the molecular entities comprising the epitope are from two or more substances and comprise a defined structure; examples include combinatorially determined epitopes associated with heterodimeric integrins. A parameter may be detection of a specifically modified protein or oligosaccharide, e.g. a phosphorylated protein, such as a STAT transcriptional protein; or sulfated oligosaccharide, or such as the carbohydrate structure Sialyl Lewis x, a selectin ligand. The presence of the active conformation of a receptor may comprise one parameter while an inactive conformation of a receptor may comprise another. A parameter may be defined by a specific monoclonal antibody or a ligand or receptor binding determinant. Parameters may include the presence of cell surface molecules such as CD antigens (CD1-CD247), cell adhesion molecules, selectin ligands, such as CLA and Sialyl Lewis x, and extracellular matrix components. Parameters may also include the presence of secreted products such as lymphokines, including IL-2, IL-4, IL-6, growth factors, etc. (Leukocyte Typing VI, T. Kishimoto et al., eds., Garland Publishing, London, England, 1997); Chemokines in Disease: Biology and Clinical Research (Contemporary Immunology), Hebert, Ed., Humana Press, 1999. For activated T cells parameters that can be detected and/or analyzed by preferred embodiments may include IL-1R, IL-2R, IL-4R, IL-12Rβ, CD45RO, CD49E, tissue selective adhesion molecules, homing receptors, chemokine receptors, CD26, CD27, CD30 and other activation antigens. Additional parameters that are modulated during activation include MHC class II; functional activation of integrins due to clustering and/or conformational changes; T cell proliferation and cytokine production, including chemokine production. Of particular importance is the regulation of patterns of cytokine production, the best-characterized example being the production of IL-4 by Th2 cells, and interferon-γ by Th1 T cells.

In an exemplary embodiment, the device can detect and/or analyze the sudden appearance (or a large increase in the local concentration) of a high affinity analyte. Serial or continuous readings would establish a background rate, against which an increase in slope would be readily detectable. The first effect of administering an effective chemotherapeutic agent might be a sudden release of otherwise bound surface and intracellular antigens unique to the malignant cells as those cells were killed. The sensor could act like a sponge for those antigens. Continuous readings from the sensing device would show a sudden change in the rate of absorption of the antigen. This could be useful as a research and clinical tool to screen the efficacy of a battery of putative chemotherapeutic agents.

An exemplary embodiment of the sensing device can be adapted to detect and/or analyze the delivery and/or concentration of chemotherapy agents in various areas of the body, including but not limited to tumors, organs, lymph nodes and other tissues of interest. For example, in an exemplary embodiment the biochemical sensor can be adapted to detect and/or determine the quantity of Taxol in the vicinity of a tumor. In the exemplary embodiment, the biosensor material 116 contains tubulin, a protein that binds to Taxol. The tubulin can be stabilized, and can be covalently bound to PEG or another substrate. The biosensor device is placed into a tumor, and fluorescently-labeled Taxol can be delivered into the patient. The fluorescently-labeled Taxol then binds to the attached tubulin in the biosensor material 116, and the fluorescence is detected and analyzed. In another exemplary embodiment, the Taxol that is delivered into the patient comprises a known ratio of fluorescently-tagged to non-fluorescently-tagged Taxol. For example, in an embodiment five non-fluorescently-tagged Taxol molecules are delivered into the patient for every one fluorescently-tagged Taxol molecule. Interstitial levels of Taxol in the vicinity of the tumor could be measured relatively by determining the fluorescence of the fluorescently tagged Taxol bound to the tubulin in the biosensor material 116. Various ratios of Taxol and/or different chemotherapy agents can be analyzed using the system described in this exemplary embodiment. For example, the biosensor material 116 can comprise tubulin dimers covalently bound to dextran, and fluorescently-labeled vincristine can be delivered with non-fluorescently-labeled vincristine to determine the circulating levels of vincristine in the vicinity of the tumor.

In another embodiment, the sensing device could be adapted to detect the enzymes responsible for control the genomic and proteomic machinery associated with apoptosis. Detection of such enzymes could be useful as an indicator of cell death, which could be used to determine the efficacy of chemotherapies. Additional biomolecules associated with cancer cells and apoptosis are known to those skilled in the art, and can be detected using alternative embodiments of the sensors. For example, molecular cascades, chemotherapy agents, and apoptosis are discussed in the following references, the contents of which are all incorporated by reference: Kim et al. (2002) “Current status of the molecular mechanisms of anticancer drug-induced apoptosis,” Cancer Chemother. Pharmacol. 50:343-352; Haupt et al. (2003) “Apoptosis—the p53 network,” J. Cell Sci. 116:4077-4085; Denmeade & Isaacs (2004) “Programmed Cell Death (Apoptosis) and Cancer Chemotherapy,” Cancer Control Journal, vol. 3, no. 4, 9 pp; and Kasili et al. (2004) “Optical sensor for the detection of caspase-9 activity in a single cell,” J. Am. Chem. Soc. 126:2799-2806.

In some embodiments used to detect analytes associated with apoptosis, the biochemical sensor could be somewhat larger and/or lower in its permeability than sensors targeted for equilibrium assays like glucose. Equilibrium times of hours may be more desirable for detection of biomolecules associated with apoptosis. An exemplary embodiment can be configured to commence and/or cease to take measurements at certain times, depending upon the timing, sequence, and/or duration of biochemical reactions and/or sequences of interest. For example, if the time is long but highly predictable, it may be possible to give overlapping drugs and identify the active one by the precise timing of the biosensor signal with respect to the drug delivery schedule. Embodiments can also be adapted to take into account the distance and/or diffusion of apoptosis-associated biomolecules outside of the cell when measuring chemotherapy delivery and/or efficiency. The number of oligopeptide-fluorophores bound to the PEG of the sensor can be optimized in embodiments based upon numerous factors, known by those skilled in the art, which can affect biomolecular reactions. For example, in some embodiments, the higher the bound content, the longer the sensor will function against a background of biomolecular reaction activity. Rapid diffusion of the cleaved fluorophor out of the matrix reduces the background fluorescence, making it easier to detect a weak increment. Matching the diffusion time of the cleaved fluorophor to the kinetics of the surge of biomolecule release itself in the interstitital fluid could provide the largest signal-to-background.

In some situations, the insertion of the biochemical sensing device into a tumor may cause damage to tumor cells and spurious release of biomolecules to be detected, which may interfere with the invention's sensing functions. Thus, in some embodiments, the sensor could be protected by soaking it in a solution containing a nonfluorescent polypeptide target for the biomolecule to be detected, which would soak up the biomolecules that were inadvertently released by the damaged cell before they diffused out of the PEG matrix. If such inadvertent release lasts longer than the diffusion time of the sensor, a photo-activation reaction could be used. One possibility would be to bind a blocking agent onto the biomolecule substrate in the sensor's matrix using a bond that could be cleaved by UV. This bond may resist the near UV used for the photopolymerization reaction.

In some embodiments the sensor could use a competitive FRET assay based on displacement of binding between two fluorescently tagged reagents by the diffusing analyte. In other embodiments, monoclonal antibodies could be used in the sensor to achieve high affinity. In further embodiments, quantum dots can be used in the assay.

In alternative embodiments, the biosensing material comprises fluorescent reporter molecules that fluoresce upon interaction with caspase and/or other apoptosis enzymes. In some embodiments, caspase may be detected as a result of apoptosis and cell lysis, wherein the caspase is released. The caspase may cleave a polymeric segment of the fluorescent reporter molecule, thereby causing the reporter molecule to fluoresce. Fluorescent reporter molecules and related enzyme assays are described in U.S. Pat. No. 6,342,611, to Weber et al, and U.S. Pat. No. 5,698,411 to Lucas et al., both of which are hereby incorporated by reference.

In alternative embodiments, the biosensor element can be adapted to enter individual healthy and/or tumor cells to detect the biomolecules of interest within the individual cells.

The previous description of the disclosed embodiments is provided to enable any person skilled in the art to make or use the biochemical sensing devices and methods. Various modifications to these embodiments will be readily apparent to those skilled in the art, and the generic principles defined herein may be applied to other embodiments without departing from the spirit or scope of the biochemical sensing devices and methods. Thus, the biochemical sensing devices and methods are not intended to be limited to the embodiments shown herein but is to be accorded the widest scope consistent with the principles and novel features disclosed herein.

What is claimed is:

1. A device for detecting an analyte from within a patient's body, comprising:
an optical fiber having:
a first end; and
a second end configured to connect to an analyzer; and
a biosensing material attached to the first end comprising:
a polymer matrix; and
tubulin, that binds with a chemotherapeutic agent, covalently bound to the polymer matrix.

2. The device of claim 1, wherein the polymer matrix comprises polyethylene glycol.

3. The device of claim 1, wherein the first end of the optical fiber further comprises a chemically altered adhesion region to which the biosensing material is attached.

4. The device of claim 1, wherein the first end of the optical fiber further comprises a mechanically altered adhesion region to which the biosensing material is attached.

5. The device of claim 1, wherein the chemotherapeutic agent comprises a mitotic inhibitor.

6. A device for detecting an analyte from within a patient's body, comprising:
an optical fiber having:
a first end; and
a second end configured to connect to an analyzer; and
a biosensing material attached to the first end comprising:
a polymer matrix; and
at least one fluorescent molecule covalently bound to the polymer matrix that fluoresces upon binding with at least one molecule indicative of apoptosis.

7. The device of claim 6, wherein the polymer matrix comprises polyethylene glycol.

8. The device of claim 6, wherein the biosensing material comprises a plurality of fluorescent molecules that can fluoresce upon interaction with at least one molecule indicative of apoptosis.

9. The device of claim 6, wherein the first end of the optical fiber further comprises a chemically altered adhesion region to which the biosensing material is attached.

10. The device of claim 6, wherein the first end of the optical fiber further comprises a mechanically altered adhesion region to which the biosensing material is attached.

11. The device of claim 6, wherein the at least one molecule indicative of apoptosis comprises caspase.

12. A method of detecting the effectiveness of a chemotherapy agent, comprising:
a) implanting an optical fiber having an implanted and free end within the patient's body such that the implanted end lies within a tumor and the free end protrudes from the patient's body, wherein the implanted end comprises biosensing material;
b) delivering a chemotherapy agent to the patient's body;
c) delivering light from an analyzer to the biosensing material through the optical fiber;
d) receiving light from the biosensing material through the optical fiber; and
e) analyzing the light that returns to the analyzer from the biosensing material;
wherein the biosensing material comprises:
a polymer matrix; and
at least one fluorescent molecule covalently bound to the polymer matrix and that fluoresces upon interaction with at least one molecule indicative of apoptosis.

13. The method of claim 12, wherein the polymer matrix comprises polyethylene glycol.

14. The method of claim 12, wherein the optical fiber further comprises a chemically altered adhesion region to which the biosensing material is attached.

15. The method of claim 12, wherein the optical fiber further comprises a mechanically altered adhesion region to which the biosensing material is attached.

16. The device of claim 12, wherein the at least one molecule indicative of apoptosis comprises caspase.
